SEQUENCE LISTING (7 sequences)

SEQ ID NO: 1 - DNA, 1094 bp, Homo sapiens
  CDS (3)..(623)
ccatgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagacagggctgccactccccaccaccgtccccagccccgttctgttccgggctgggactctgcccccggagcaccctccccagctgacatcacccatcccactccagccccaggcccctctgcccacgctgcacccagcaccaccagcgccctgacccccggacctgccgctgccgctgccgacgccgcagcttcctccgttgccaagggcggggcttagagctcaacccagacacctgcaggtgccggaagctgcgaaggtgacacatggcttttcagactcagcagggtgacttgcctcagaggctatatcccagtgggggaacaaaggggagcctggtaaaaaacagccaagcccccaagacctcagcccaggcagaagctgctctaggacctgggcctctcagagggctcttctgccatcccttgtctccctgaggccatcatcaaacaggacagagttggaagaggagactgggaggcagcaagaggggtcacataccagctcaggggagaatggagtactgtctcagtttctaaccactctgtgcaagtaagcatcttacaactggctcttcctcccctcactaagaagacccaaacctctgcataatgggatttgggctttggtacaagaactgtgacccccaaccctgataaaagagatggaaggaaaaaaaaaaaaaaa

SEQ ID NO: 2 - protein, 207 aa, Homo sapiens
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 3 - DNA, 993 bp, Homo sapiens
  CDS (3)..(566)
ccatgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagatagccccaggcccctctgcccacgctgcacccagcaccaccagcgccctgacccccggacctgccgctgccgctgccgacgccgcagcttcctccgttgccaagggcggggcttagagctcaacccagacacctgcaggtgccggaagctgcgaaggtgacacatggcttttcagactcagcagggtgacttgcctcagaggctatatcccagtgggggaacaaaggggagcctggtaaaaaacagccaagcccccaagacctcagcccaggcagaagctgctctaggacctgggcctctcagagggctcttctgccatcccttgtctccctgaggccatcatcaaacaggacagagttggaagaggagactgggaggcagcaagaggggtcacataccagctcaggggagaatggagtactgtctcagtttctaaccactctgtgcaagtaagcatcttacaactggctcttcctcccctcactaagaagacccaaacctctgcataatgggatttgggctttggtacaagaactgtgacccccaaccctgataaaagagatggaaggaaaaaaaaaaaaaaa

SEQ ID NO: 4 - protein, 188 aa, Homo sapiens
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDSPRPLCPRCTQHHQRPDPRTCRCRCRRRSFLRCQGRGLELNPDTCRCRKLRR

SEQ ID NO: 5 - DNA, 18 bp, Artificial Sequence
  synthetic primer
tttgatggccccagccac

SEQ ID NO: 6 - DNA, 18 bp, Artificial Sequence
  synthetic primer
cccccagctgactgctcg

SEQ ID NO: 7 - DNA, 18 bp, Artificial Sequence
  synthetic primer
ctagtggatcccccgggc

(19) United States
(12) Patent Application Publication
(10) Pub. No.: US 2004/0115166 A1
(43) Pub. Date: Jun. 17, 2004

(54) Method of treatment and prophylaxis

(76) Inventors: Nick Kim Hayward, Queensland (AU); Graham Frederick Kay, Queensland (AU); Arne Wyndham Mould, Queensland (AU)

Correspondence Address: Frank S DiGiglio, Scully Scott Murphy & Presser, 400 Garden City Plaza, Garden City, NY, 11530, US

(21) Appl. No.: 10/220,324

(22) PCT Filed: Feb. 28, 2001
(86) PCT No.: PCT/AU01/00205

(30) Foreign Application Priority Data
Feb. 29, 2000 (AU) PQ5921

(51) Int. Cl. A61K 38/19; A61K 38/18

(52) U.S. Cl. 424/85.1; 514/12

(57) ABSTRACT

The present invention relates generally to a method for the treatment and/or prophylaxis of a disease condition, agents useful in such treatments and an animal model useful in screening and evaluating potentially efficacious therapeutic agents. More particularly, the present invention contemplates a method for the treatment and/or prophylaxis of a local or systemic autoimmune condition such as but not limited to rheumatoid arthritis or a related condition. The method of the present invention is predicted in part on the determination that the onset and/or severity of particular disease conditions is exacerbated or otherwise facilitated by certain growth factors or cytokines. Temporary or sustained reduction in the levels of these growth factors or cytokines is shown to reduce the onset and/or severity and/or to otherwise ameliorate the conditions of the disease conditions.

### FIELD OF THE INVENTION

The present invention relates generally to a method for the treatment and/or prophylaxis of a disease condition, agents useful in such treatments and an animal model useful in screening and evaluating potentially efficacious therapeutic agents. More particularly, the present invention contemplates a method for the treatment and/or prophylaxis of a local or systemic autoimmune condition such as but not limited to rheumatoid arthritis or a related condition. The method of the present invention is predicated in part on the determination that the onset and/or severity of particular disease conditions is exacerbated or otherwise facilitated by certain growth factors or cytokines. Temporary or sustained reduction in the levels of these growth factors or cytokines is shown to reduce the onset and/or severity and/or to otherwise ameliorate the conditions of the disease conditions.

### BACKGROUND OF THE INVENTION

Reference to any prior art in this specification is not, and should not be taken as, an acknowledgment or any form of suggestion that this prior art forms part of the common general knowledge in Australia or any other country.

Bibliographic details of the publications referred to by author in this specification are collected at the end of the description.

The increasing sophistication of recombinant DNA technology is greatly facilitating research and development in the medical and allied health industries. Of particular relevance is the ability to produce laboratory test animals which are substantially incapable of or which otherwise have reduced capacity to express a particular gene or genetic sequence. Such animals are generally referred to as “knockout” animals. The development of such animals provides a model for assessing the development and progression of disease conditions within a particular genetic background.

A vast number of growth factors and cytokines have been identified. Whilst many of those molecules are required to prevent the development of a disease condition, certain disease conditions are exacerbated by even normal levels of a particular growth factor or cytokine.

The vascular endothelial growth factors and their receptors are important molecules and provide a potential source of therapeutic and diagnostic agents for conditions characterized by defective or aberrant angiogenesis (Olofsson et al., 1999). One particular growth factor is vascular endothelial growth factor B (VEGF-B). VEGF-B is abundant in heart and skeletal muscle and aberrations in the molecule or its encoding gene may be associated with vascular malformations and/or cardiovascular disease (Bellomo et al., 2000; Makinen et al., 1999; Aase et al., 1999; Paavonen et al., 1996). The VEGF-B molecule has been purified to homogeneity and genetic sequences encoding VEGF-B have been cloned from both human (Grimmond et al., 1996) and from murine (Townson et al., 1996) sources. The human gene encoding VEGF-B is denoted herein “VEGFB”. Its two splice isoforms encode VEGF-B₁₆₇ and VEGF-B₁₈₆. Reference herein to VEGFB includes homologues of VEGFB including other mammalian homologues. The murine orthologue of VEGFB is denoted Vegfb. This is also regarded as a murine homologue of VEGFB.

VEGF-B is, therefore, an important molecule making it a potentially valuable target for the development of therapeutics, prophylactics and diagnostic agents based on VEGF-B or its activities.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1, <400>2, etc. A sequence listing is provided after the claims.

Accordingly, one aspect of the present invention contemplates a method for the treatment and/or prophylaxis of a disease condition in a subject wherein said disease condition is an autoimmune condition exacerbated or otherwise facilitated by the presence of a growth factor or cytokine or expressible genetic material encoding a growth factor or cytoline and/or genetic material which facilitates the expression of said first mentioned genetic material said method comprising reducing or inhibiting the level or activity of the growth factor or cytokine or reducing or inhibiting the expression or function of genetic material encoding said growth factor or cytokine for a time and under conditions sufficient to delay onset of or to otherwise ameliorate the symptorns of said disease condition.

Another aspect of the present invention provides a method for the treatment and/or prophylaxis of rheumatoid arthritis or a related condition in a subject wherein said rheumatoid arthritis or related condition is a condition exacerbated or otherwise facilitated by the presence of a growth factor or cytokine or expressible genetic material encoding a growth factor or cytoline and/or genetic material which facilitates the expression of said first mentioned genetic material said method comprising reducing or inhibiting the level or activity of the growth factor or cytokine or reducing or inhibiting the expression or function of said genetic material encoding said growth factor or cytokine for a time and under conditions sufficient to delay onset of or otherwise ameliorate the symptoms of said rheunatoid arthritis or related condition.

Yet another aspect of the present invention is directed to a method for the prophylaxis and/or treatment of an autoimmune condition or a related condition, said method comprising reducing the level or activity of VEGF-B or a functional or structural equivalent thereof or reducing or inhibiting the function of genetic material encoding VEGF-B or which facilitates expression of VEGFB or its homologue for a time and under conditions sufficient to reduce onset of or otherwise ameliorate the symptoms of an autoimmnune disease or a related condition.

Still another aspect of the present invention is directed to a method for the prophylaxis and/or treatment of rheumatoid arthritis or a related condition, said method comprising reducing the level or activity of VEGF-B or a functional or structural equivalent thereof or reducing or inhibiting the finction of genetic material encoding VEGF-B or which facilitates expression of VEGFB or its homologue for a time and under conditions sufficient to reduce onset of or otherwise ameliorate the symptoms of rheumatoid arhritis or a related condition.

A further aspect of the present invention contemplates the use of a VEGF-B level- or activity-inhibiting or antagonizing molecule in the manufacture of a medicarnent for the treatment of an autoimmune condition such as rheumatoid arthritis or related condition.

Still a further aspect of the present invention provides for the use of a VEGFB- or VEGFB homologue- or associated regulatory sequence-expression inhibiting or antagonizing molecule in the manufacture of a medicament for the treatment of an autoimmune condition such as rheumatoid arthritis or related condition.

Still yet another aspect of the present invention provides, therefore, a composition comprising an antagonist of growth factor or cytokine activity or antagonists of expression of genetic sequences encoding the growth factor or cytokine and one or more pharmaceutically acceptable carriers and/or diluents.

Another aspect of the present invention provides a composition comprising a VEGF-B or VEGFB or VEGFB homologue antagonist and one or more pharmaceutically acceptable carriers and/or diluents for use in the prophylaxis and/or treatment of an autoimmune condition.

Yet another aspect of the present invention provides a genetically modified animal wherein said animal produces a greater amount of a growth factor or cytokine relative to a non-genetically modified animal of the same species wherein said animal has a predisposition for the development of an autoimmune condition.

Still another aspect of the present invention provides a genetically modified mouse wherein said mouse produces a greater amount of a growth factor or cytokine relative to a non-genetically modified mouse of the same strain wherein said mouse has a predisposition for the development of an autoimmune condition.

A further aspect of the present invention provides a genetically modified animal wherein said animal is substantially incapable of producing a growth factor or cytokine relative to a non-genetically modified animal of the same species wherein said animal has a reduced onset or reduced clinical severity of an autoimmune condition.

Still another aspect of the present invention provides a genetically modified mouse wherein said mouse is substantially incapable of producing a growth factor or cytokine relative to a non-genetically modified mouse of the same strain wherein said mouse has a reduced onset or reduced clinical severity of an autoimmune condition.

A further aspect of the present invention provides a targeting vector useful for inactivating a gene encoding a growth factor or cytokine, said targeting vector comprising two segments of genetic material encoding said growth factor or cytokine flanking a positive selectable marker wherein when said targeting vector is transfected into embryonic stem (ES) cells and the marker selected, an ES cell is generated in which the gene encoding said growth factor or cytokine is inactivated by homologous recombination.

Still a further aspect of the present invention provides a targeting vector useful for inactivating a gene encoding VEGF-B or other growth factor or cytokine, said targeting vector comprising two segments of genetic material encoding VEGF-B or other growth, factor or cytokine flanking a positive selectable marker wherein when said targeting vector is transfected into embryonic stem (ES) cells and the marker selected, an ES cell is generated in which the Vegfb or other gene encoding said other growth factor or cytokine is inactivated by homologous recombination.

Still yet another aspect of the present invention is directed to the use of a targeting vector as defined above in the manufacture of a genetically modified animal substantially incapable of producing VEGF-B or other growth factor or cytokine.

Another aspect of the present invention is directed to the use of a targeting vector as defined above in the manufacture of a genetically modified mouse substantially incapable of producing VEGF-B or other growth factor or cytokine.

### BRIEF DESCRIPTION OF THE FIGURES

FIG. 1 is a diagrammatic representation of the murine Vegfb gene (top), the targeting construct used to generate a Vegfb^{+/-} mouse (middle) and the final targeted locus (bottom). The exons of the Vegfb gene are shown as numbered boxes with the open reading frame as open boxes. The location and orientation of the PCR primers used to genotype mice are shown as PCR1, PCR2 and PCR3. The location and orientation of the Southern blot probes used to genotype mice are shown as Probe 1 and Probe 2.

FIG. 2 is a graphical representation showing the development of rheumatoid arthritis in Vegfb knockout female mice. —C— Vegfb^{+/+} (n=12); —a— Vegfb^{+/-} (n=17); —B— Vegfb^{-/-} (n=10). The “*” indicates a significant difference where P<0.05 when compared with Vegfb knockout mice.

FIG. 3 is a graphical representation showing incidence of rheumatoid arthritis in Vegfb knockout female mice. —C— Vegfb^{+/+} (n=12); - -^{a}- - Vegfb^{+/-} (n=17); —B— Vegfb^{-/-} (n=10).

FIG. 4 is a graphical representation showing the development of rheumatoid arthritis in Vegfb knockout male mice. —C— Vegfb^{+/+} (n=14); —B— Vegfb^{-/-} (n=9). The “*” indicates a significant difference where P<0.05 when compared with Vegfb knockout mice.

FIG. 5 is a graphical representation showing the incidence of rheumatoid arthritis in Vegfb knockout male mice. —C— Vegfb^{+/+} (n=14); —B— Vegfb^{-/-} (n=9).

### DETAILED DESCRIPTION OF THE PREFERRED EMBODIMENTS

In accordance with the present invention, the inventors determined that rheumatoid arthritis exhibits most rapid onset and severity within a genetic background comprising Vegfb in homozygous or heterozygous form. This finding permits the development of therapeutic protocols for autoimmnune and related disease conditions in human and other mammalian subjects by the local or systemic reduction of a particular growth factor or cytokine or of expression of genetic material encoding the growth factor or cytokine which growth factor or cytoline exacerbates or otherwise facilitates the disease condition.

Accordingly, one aspect of the present invention contemplates a method for the treatment and/or prophylaxis of a disease condition in a subject wherein said disease condition is an autoimmune condition exacerbated or otherwise facilitated by the presence of a growth factor or cytokine or expressible genetic material encoding a growth factor or cytokine and/or genetic material which facilitates the expression of said first mentioned genetic material said method comprising reducing or inhibiting the level or activity of the growth factor or cytokine or reducing or inhibiting the expression or function of genetic material encoding said growth factor or cytokine for a time and under conditions sufficient to delay onset of or to otherwise ameliorate the symptoms of said disease condition.

Optionally, sequentially or simultaneously to reducing the expression or function of the genetic material, a further treatment protocol may be instituted to ameliorate the symptoms of the disease condition being treated.

Reference herein to “sequentially” means that two or more treatments occur within seconds, minutes, hours, days, weeks or months of each other. “Simultaneously” includes the co-treatment at substantially the same time.

The method of the present invention may be practised systemically or locally. For example, if the disease condition affects a particular part of the body such as joints, organs or skin, the growth factor or cytokine may only need to be reduced at that location. This is referred to herein as a “local” reduction in the growth factor or cytokine. When the growth factor or cytokine needs to be reduced in the entire body or in a substantial part of the body, this is referred to as “systemic” reduction. The reduction may be permanent or semi-permanent including temporary. A temporary reduction includes a reduction of up to minutes, hours, days or months.

The present invention encompasses any autoimmune condition such as but not limited to rheumatoid arthritis, ankylosing spondylitis, acute anterior uveitis, Goodpastures's syndrome, multiple sclerosis, Graves' disease, myasthenia gravis, systemic lupis erythematosus, insulin-dependent diabetes mellitus, pemphigus vulgaris, Hashimoto's thyroiditis, autoimmune hemolytic anemia, autoimmune thrombocytopenia purpura, acute rheumatic fever, subacute bacterial endocarditis, mixed essential cryoglobulinemia, experimental autoimmune encephalomyelitis (EAE), hypoglycemia and cold agglutinin disease.

The most preferred autoimnune condition is rheumatoid arthritis or a related condition. A “related condition” is a condition which comprises symptoms, etiologies, outcomes and prognoses similar to rheumatoid arthritis. A related condition may not, however, have the same physiological basis as rheumatoid arthritis. Accordingly, a condition related to rheumatoid arthritis may not necessarily be an autoimmune disease. The present invention is hereinafter disclosed with reference to rheumatoid arthritis and related conditions. This is done, however, with the understanding that the present invention extends to the treatment and/or prophylaxis of any autoinmune disease condition which is exacerbated or otherwise facilitated by a growth factor or cytokine.

Accordingly, another aspect of the present invention provides a method for the treatment and/or prophylaxis of rheumatoid arthritis or a related condition in a subject wherein said rheumatoid arthritis or related condition is a condition exacerbated or otherwise facilitated by the presence of a growth factor or cytokine or expressible genetic material encoding a growth factor or cytokine and/or genetic material which facilitates the expression of said first mentioned genetic material said method comprising reducing or inhibiting the level or activity of the growth factor or cytokine or reducing or inhibiting the expression or function of said genetic material encoding said growth factor or cytokine for a time and under conditions sufficient to delay onset of or otherwise ameliorate the symptoms of said rheumatoid arthritis or related condition.

The preferred growth factor or cytokine in accordance with the present invention is VEGF-B. The gene encoding VEGF-B, i.e. VEGFB, may be subject to deletion or mutagenesis or expression of VEGFB may be reduced using such means as but not limited to ribozymes, antisense molecules and co-suppression. Furthermore, genetic sequences which are required for expression or processing of VEGFB may be the target. An example of such a genetic sequence is a regulatory or promoter region or a region involved in splicing. Alternatively, or in addition to, the activity of the VEGF-B protein may be reduced using such means as but not limited to antagonists, inhibitory peptides or chemical molecules, antibodies or soluble VEGF-B receptors or homologues or analogues thereof. The level of VEGF-B protein may also be reduced using soluble receptors or homologues or analogues thereof or antibodies against other means.

In a preferred embodiment, VEGF-B is naturally occurring VEGF-B or its recombinant equivalent. However, the present invention extends to homologues and functional and structural equivalents of VEGF-B. A “functional equivalent” includes another VEGF-B species or related molecule which exacerbates or facilitates an autoimmnune disease such as rheumatoid arthritis or related condition. An example of a functional equivalent includes a derivative comprising amino acids 10-108 of VEGFB sequences shown in SEQ ID NOS:2 and 4. Reference herein to “VEGF-B” includes splice variants and other mutants and derivatives of VEGF-B.

The Genbank accession number for human nucleotide and amino acid sequences for VEGF-B are U43368, U43369 and U43370. The murine sequences for Vegfb are represented in U43836 and U43837. Particular VEGFB nucleotide sequences are referred to as VEGFB₁₈₆ (SEQ ID NO:1) and VEGFB₁₆₇ (SEQ ID NO:3). Amino acid sequences for VEGF-B₁₈₆ and VEGF-B₁₆₇ are shown in SEQ ID NO:2 and SEQ ID. NO:4, respectively.

Accordingly, another aspect of the present invention is directed to a method for the prophylaxis and/or treatment of an autoimmune condition or a related condition, said method comprising reducing the level or activity of VEGF-B or a functional or structural equivalent thereof or reducing or inhibiting the function of genetic material encoding VEGF-B or which facilitates expression of VEGFB for at time and under conditions sufficient to reduce onset of or otherwise ameliorate the symptoms of an autoimmune disease or a related condition.

More particularly, the present invention is directed to a method for the prophylaxis and/or treatment of rheumatoid arthritis or a related condition, said method comprising reducing the level or activity of VEGF-B or a functional or structural equivalent thereof or reducing or inhibiting the function of genetic material encoding VEGF-B or which facilitates expression of VEGFB or its homologue for at time and under conditions sufficient to reduce onset of or otherwise ameliorate the symptoms of rheumatoid arthritis or a related condition.

The instant method may also require the simultaneous or sequential practice of one or more other therapeutic protocols useful in the treatment and/or prophylaxis of rheumatoid arthritis or a related condition.

Reference to a “subject” includes reference to any animal and more particularly to any mammal such as but not limited to a human, primate, laboratory test animal (e.g. mouse, rat, rabbit, guinea pig, hamster), livestock animal (e.g. sheep, cow, pig, horse, donkey), companion animal (e.g. cat, dog) or captive wild animal. Although a human is a particularly preferred subject, the prevention and/or treatment of rheumatoid arthritis or related condition is also important in the veterinary field and is encompassed by the present invention.

The practice of the present invention may be directed at either male or female mammals although in laboratory test animals, female animals exhibited reduced clinical severity of rheumatoid arthritis at early and late stages whereas male animals exhibited reduced rheumatoid arthritis at early and late stages whereas male animals exhibited reduced clinical onset of rheumatoid arthritis but generally not a reduction in clinical severity at later stages of the disease.

The practice of the present invention is preferably by subjecting a human or animal patient to VEGF-B level- or activity-reduction means or VEGFB- or its homologue- expression reduction means. Accordingly, the present invention extends to compositions comprising antagonists, antibodies, chemical inhibitor molecules, antisenge molecules, co-suppression molecules and/or ribozymes or any other means for reducing the level or activity of VEGF-B or the expression of VEGFB or its homologue or associated regulatory sequences.

The present invention further contemplates the use of a VEGF-B level- or activity-inhibiting or antagonizing molecule in the manufacture of a medicament for the treatment of an autoimmnune condition such as rheumatoid arthritis or related condition.

In a related aspect of the present invention, the present invention provides for the use of VEGFB- or VEGFB homologue- or associated regulatory sequence-expression inhibiting or antagonizing molecule in the manufacture of a medicament for the treatment of an autoimmune condition such as rheumatoid arthritis or related condition.

The present invention provides, therefore, a composition comprising an antagonist of growth factor or cytoline activity or antagonists of expression of genetic sequences encoding the growth factor or cytoline and one or more pharmaceutically acceptable carriers and/or diluents.

Preferably, the growth factor or cytokine is VEGF-B.

Accordingly, in a particularly preferred embodiment, there is provided a composition comprising a VEGF-B or VEGFB or VEGFB homologue antagonist and one or more pharmaceutically acceptable carriers and/or diluents for use in the prophylaxis and/or treatment of an autoimmune condition. Preferably, the autoimmune condition is rheumatoid arthritis or a related condition. Most preferably, the autoimmune disease is rheumatoid arthritis.

The composition may also be referred to as a pharmaceutical composition. The composition may also be regarded as an agent.

The composition of this aspect of the present invention may also comprise one or more other medicaments useful in the treatment of, for example, rheumatoid arthritis or a related or associated condition.

The composition may be adapted or in a form for use topically, locally or systemically.

When the active ingredient is suitably protected, it may be orally administered, for example, with an inert diluent or with an assimilable edible carrier, or it may be enclosed in hard or soft shell gelatin capsule, or it may be compressed into tablets.

Pharmaceutically acceptable carriers and/or diluents include any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, use thereof in the therapeutic compositions is contemplated. Supplementary active ingredients can also be incorporated into the compositions.

Methods and pharmaceutical carriers for preparation of pharmaceutical compositions are well known in the art, as set out in textbooks such as Remington's Pharmaceutical Sciences, 17th Edition, Mack Publishing Company, Easton, Pa. USA.

Yet another aspect of the present invention provides an animal model useful for screening for agents capable of ameliorating the effects of an autoimmune condition such as rheumatoid arthritis. In one embodiment, the animal model produces excess amounts of the growth factor or cytokine such as but not limited to VEGF-B. Such an animal would have a predisposition for either developing an autoimmune condition such as rheumatoid arthritis or related condition or would readily develop the condition following immnunization or treatment with an autoimmune disease-inducing agent. Such an animal model is usefil for screening for agents which inhibit or ameliorate the conditions associated with, for example, rheumatoid artritis or related condition.

Accordingly, another aspect of the present invention provides a genetically modified animal wherein said animal produces a greater amount of a growth factor or cytokine relative to a non-genetically modified animal of the same species wherein said animal has a predisposition for the development of an autoimmune condition.

Preferably, the genetically modified animal is a mouse, rat, guinea pig, rabbit, pig, sheep or goat. More preferably, the genetically modified animal is a mouse or rat. Most preferably, the genetically modified animal is a mouse.

Accordingly, a preferred aspect of the present invention provides a genetically modified mouse wherein said mouse produces a greater amount of a growth factor or cytokine relative to a non-genetically modified mouse of the same strain wherein said mouse has a predisposition for the development of an autoimmune condition.

Another animal model contemplated by the present invention comprises an animal which is substantially incapable of producing a particular growth factor or cytokine such as VEGF-B. Generally, but not exclusively, such an animal is referred to as a homozygous or heterozygous Vegfb-knockout animal. Such animals have reduced onset and/or reduced clinical severity of for example, rheumatoid arthritis. These animals are useful for screening for naturally occurring agents such as growth factors and cytokines other than VEGF-B which also have the effect of inducing or facilitating the onset or clinical severity of the disease condition such as rheumatoid arthritis. Once such molecules are identified, a treatment protocol can be developed which targets not only, for example, VEGF-B or VEGFB, but also any other endogenous molecules which might also be associated with the development of the autoimmune disease such as rheumatoid arthritis or related condition.

According to this aspect of the present invention, there is provided a genetically modified animal wherein said animal is substantially incapable of producing a growth factor or cytokine relative to a non-genetically modified animal of the same species wherein said animal has a reduced onset or reduced clinical severity of an autoimmnune condition.

Preferably, the genetically modified animal is a mouse, rat, guinea pig, rabbit, pig, sheep or goat. More preferably, the genetically niodified animal is a mouse or rat. Most preferably, the genetically modified animal is a mouse.

According to this aspect, there is provided a genetically modified mouse wherein said mouse is substantially incapable of producing a growth factor or cytokine relative to a non-genetically modified mouse of the same strain wherein said mouse has a reduced onset or reduced clinical severity of an autoimmune condition.

The autoimmune conditions contemplated by these animal models include rheumatoid arthritis, ankylosing spondylitis, acute anterior uveitis, Goodpastures's syndrome, multiple sclerosis, Graves' disease, myasthenia gravis, systemic lupis erythematosus, insulin-dependent diabetes mellitus, pemphigus vulgaris, Hashimoto's thyroiditis, autoimmune hemolytic anemia, autoimmune thrombocytopenia purpura, acute rheumatic fever, subacute bacterial endocarditis, mixed essential cryoglobulinemia, experimental autoimmune encephalomyelitis (EAE), hypoglycemia and cold agglutinin disease.

Preferably, the autoimmune condition is rheumatoid arthritis or a related condition. Most preferably, the autoimmune condition is rheumatoid arthritis.

The animal models of the present invention may be in the form of the animals or may be, for example, in the form of embryos for transplantation. The embryos arc preferably maintained in a frozen state and may optionally be sold with instructions for use.

Yet another aspect of the present invention provides a targeting vector useful for inactivating a gene encoding a growth factor or cytokine, said targeting vector comprising two segments of genetic material encoding said growth factor or cytokine flanking a positive selectable marker wherein when said targeting vector is transfected into embryonic stem (ES) cells and the marker selected, an ES cell is generated in which the gene encoding said growth factor or cytokine is inactivated by homologous recombination.

Preferably, the growth factor or cytokine is VEGF-B.

Still another aspect of the present invention provides a targeting vector useful for inactivating a gene encoding VEGF-B or other growth factor or cytokine, said targeting vector comprising two segments of genetic material encoding VEGF-B or other growth factor or cytoline flanking a positive selectable marker wherein when said targeting vector is transfected into embryonic stem (ES) cells and the marker selected, an ES cell is generated in which the Vegfb or other gene is inactivated by homologous recombination.

Preferably, the ES cells are from rnice, rats, guinea pigs, pigs, sheep or goats. Most preferably, the ES cells are from mice.

Still yet another aspect of the present invention is directed to the use of a targeting vector as defined above in the manufacture of a genetically modified animal substantially incapable of producing VEGF-B or other growth factor or cytokine.

Even still another aspect of the present invention is directed to the use of a targeting vector as defined above in the manufacture of a genetically modified mouse substantially incapable of producing VEGF-B or other growth factor or cytokine.

Preferably, the vector is DNA. A selectable marker in the targeting vector allows for selection of targeted cells that have stably incorporated the targeting DNA. This is especially useful when employing relatively low efficiency transformation techniques such as electroporation, calcium phosphate precipitation and liposome fusion where typically fewer than 1 in 1000 cells will have stably incorporated the exogenous DNA. Using high efficiency methods, such as microinjection into nuclei, typically from 5-25% of the cells will have incorporated the targeting DNA; and it is, therefore, feasible to screen the targeted cells directly without the necessity of first selecting for stable integration of a selectable marker.

Examples of selectable markers include genes conferring resistance to compounds such as antibiotics, genes conferring the ability to grow on selected substrates, genes encoding proteins that produce detectable signals such as luminescence. A wide variety of such markers are known and available, including, for example, antibiotic resistarice genes such as the neomycin resistance gene (neo) [Southern and Berg, 1982] and the hygromycin resistance gene (hyg) [Te Riele et al., 1990]. Selectable markers also include genes conferring the ability to grow on certain media substrates such as the tk gene (thyrnidine kinase) or the hprt gene (hypoxanthine phosphoribosyltransferase) which confer the ability to grow on HAT medium (hypoxanthine, aminopterin and thymidine); and the bacterial gpt gene (guanine/xanthine phosphoribosyltransferase) which allows growth on MAX medium (mycophenolic acid, adenine and xanthine). See Song et al. (1987). Other selectable markers for use in mammalian cells and plasmids carrying a variety of selectable markers are described in Sambrook et al. (1989).

The preferred location of the marker gene in the targeting construct will depend on the aim of the gene targeting. For example, if the aim is to disrupt target gene expression, then the selectable marker can be cloned into targeting DNA corresponding to coding sequence in the target DNA Alternatively, if the aim is to express an altered product from the target gene, such as a protein with an amino acid substitution, then the coding sequence can be modified to code for the substitution, and the selectable marker can be placed outside of the coding region, for example, in a nearby intron.

The selectable marker may depend on its own promoter for expression and the marker gene may be derived from a very different organism than the organism being targeted (e.g. prokaryotic marker genes used in targeting mammalian cells). However, it is preferable to replace the original promoter with transcriptional machinery known to function in the recipient cells. A large number of transcriptional initiation regions are available for such purposes including, for example, metallothionein promoters, thymidine kinase promoters, β-actin promoters, immunoglobulin promoters, SV40 promoters and human cytomegalovirus promoters. A widely used example is the pSV2-neo plasmid which has tahe bacterial neomycin phosphotransferase gene under control of the SV40 early promoter and confers in mammnalian cells resistance to G418 (an antibiotic related to neomycin) [Southern and Berg, 1982]. A number of other variations may be employed to enhance expression of the selectable markers in animal cells, such as the addition of a poly(A) sequence (see, e.g. Thomas et al., 1986) and the addition of synthetic translation initiation sequences (see, e.g. Thomas and Capecchi, 1987). Both constitutive and inducible promoters may be used.

The DNA is preferably modified by homologous recombination. The target DNA can be in any organelle of the animal cell including the nucleus and mitochondria and can be an intact gene, an exon or intron, a regulatory sequence or any region between genes.

Homologous DNA is a DNA sequence that is at least 70% identical with a reference DNA sequence. An indication that two sequences are homologous is that they will hybridize with each other under stringent conditions (see, e.g. Sambrook et al., 1989).

Reference herein to stringent conditions includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C. The temperature may be altered and higher temperatures used to replace formainide and/or to give alternative stringency conditions. Alternative stringency conditions may be applied where necessary, such as medium stringency, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C)% (Marmur and Doty, 1962). However, the Tₘ of a duplex DNA decreases by 1° C. with every increase of 1% in the number of mismatch base pairs (Bonner and Laskey, 1974). Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

The term “homologous recombination” refers to the process of DNA recombination based on sequence homology. The term embraces both crossing over and gene conversion. Cellular recombination enzymes are believed to be involved in the process of recognizing sequence identity between distinct nucleotide sequences.

The present invention is further described by the following non-limiting Examples.

### EXAMPLE 1

### Generation of Vegfb Knockout Mice

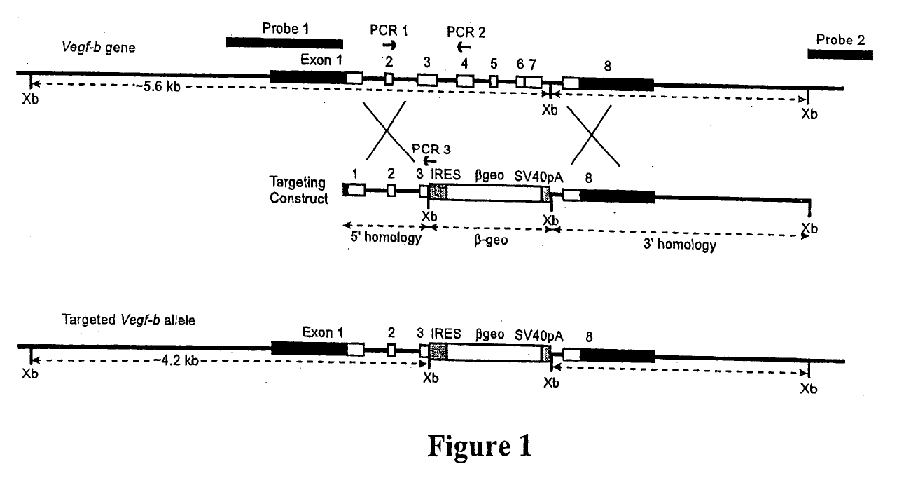

The Vegfb gene is inactivated by homologous recombination. To promote homologous recombination, a targeting vector is prepared for transfection into embryonic stem (ES) cells. In this vector, two segments of the Vegfb locus flank a suitable positive selectable marker, such as the neomycin resistance gene, neo^{r}, which renders transfected ES cells resistant to the antibiotic G418. In the case of the mice used for the work described here the selection marker used was the β-geo gene, which is a fusion between neo^{r} and lacZ. In the construct a promoter-less β-geo cassette replaced exons 3-7 of the Vegfb gene and was flanked by the remaining portion of the locus (FIG. 1). The β-geo structural gene is preceded by an internal ribosomal entry site signal sequence to give cap-independent translation of the β-geo fusion protein (Mountford et al., 1994). The resulting targeted Vegfb locus would result in expression of β-geo fusion protein under the control of the Vegfb gene promoter/enhancer rather than a finctional Vegf-B protein This targeting strategy not only renders transfected ES cells resistant to G418 but also allows for easy identification of cells capable of expression from the Vegfb locus under the control of the endogenous Vegfb promoter/enhancer. Since the introduced β-geo selection gene does not carry its own promoter its expression is reliant upon the targeting vector being either, correctly integrated by homologous recombination into the Vegfb locus, or, randomly inserted into the genome close to some other gene's promoter. This strategy greatly increases the probability that a given clone will be selected due to the correct homologous recombination targeting event.

The targeting vector is transfected by electroporation into the ES cell line which are then cultured for 7 to 10 days on mitotically inactivated mouse embryonic fibroblast (MEF) feeder cells (either γ-irradiated or mitomycin C-inactivated) in ES cell culture medium containing 10³ U/ml of leukemia inhibitory factor (LIF) to maintain the cells in an undifferentiated state. During this period G418 (200 μg/ml) is added to the culture medium to select for transfected cells which have incorporated the targeting vector and expressed the β-geo fusion protein. After selection, the resulting clones of cells are picked and cultured as individual cell lines without G418 on MEF feeder layers in ES cell culture medium with LIF, as above. Southern blotting (using Probe 1 and Probe 2 indicated in FIG. 1) or polymerase chain reaction (PCR) (using the indicated primers; PCR1, 5′-ttt gat ggc ccc agc cac-3′ (SEQ ID NO:5); PCR2, 5′-ccc cca gct gac tgc tcg-3′ (SEQ ID NO:6); PCR3, 5′-cta gtg gat ccc ccg ggc-3′ (SEQ ID NO:7) indicated in FIG. 1) is then used to identify clones that have undergone homologous recombination in the correct manner and carry the correctly targeted Vegfb locus.

Cells from clones identified as carrying the correctly targeted Vegfb locus are then microinjected into mouse blastocyst stage embryos to form chimaeras, which are subsequently surgically transferred into the uterus of pseudo-pregnant recipient female mice for development to term. Chimeric mice are identified in the newborn litters by coat colour chimerism that occurs due to the mixing of the ES cells (which, for example, carry genes resulting in agouti coat colour, e.g. 129/SvJ strain) with the cells from the host blastocyst (which, for example, carry genes resulting in black coat colour, e.g. C57BL/6J strain). Chimeric mice are then test mated to a mouse with a suitable coat colour (e.g. C57BL/6J) in order to identify those which are capable of germline transmission of the targeted Vegfb locus from their ES cell derived component. Germline transmission from the ES cell (129/SvJ strain) component of the chimaera is evident if the progeny of this mating have agouti coat colour. Germline transmission of the Vegfb targeted locus is then determined by Southern blotting or PCR of DNA, as above, derived from tail tip biopsies of the progeny with agouti coat colour. Progeny carrying the heterozygous targeted Vgfb locus (Vegfb^{+/-}) are crossed to derive homozygous Vegfb targeted mice (Vegfb^{-/-}).

Vegfb knockout mice may also be crossed for multiple generations (e.g. n=6) towards the C57BL6 mice as opposed to a smaller generation backcross mice from the 129SV to C57BL6 mice.

### EXAMPLE 2

### Development of Rheumatoid Arthritis in Female Vegfb Knockout Mice

Female Vegfb^{+/+}, Vegfb^{+/-} and Vegfb^{-/-} mice (6-8 weeks of age) were immunized with chick collagen II (CII) (100 μg) in complete Freund's adjuvant (CFA) containing 2.5 mg/ml heat inactivated M. tuberculosis on day 1 followed by a booster injection of chick CII (100 μg) in incomplete Freund's adjuvant (IFA) on day 8. Disease severity was calculated from day 23 onwards by cumulative clinical assessment (0-72) of all digits and paws, each graded on a scale of 0 to 3 where 0=normal, 1=slight swelling and/or erythema, 2=extensive swelling and/or erythema, and 3=joint distortions and/or rigidity.

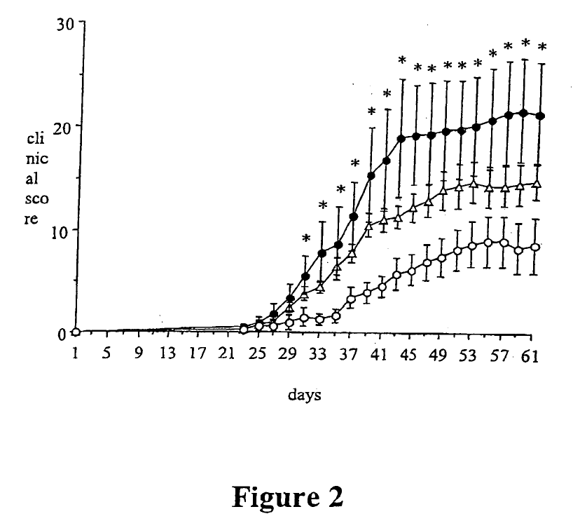

Vegfb deficient mice displayed delayed onset and reduced severity of disease. The significance of differences between experimental groups was analyzed using the alternate Welch t-test. Differences in means were considered significant if P<0.05. This is indicated by an “*” in FIG. 2.

### EXAMPLE 3

### Incidence of Rheumatoid Arthrtis in Vegfb Knockout Mice

Female Vegfb^{+/+}, Vegfb^{+/-} and Vegfb^{-/-} mice (6-8 weeks of age) were immunized with chick CII (100 μg) in CFA containing 2.5 mg/ml heat inactivated M tuberculosis on day 1 followed by a booster injection of chick CII (100 μg) in IFA on day 8. Disease incidence was determined as the percentage of animals exhibiting clinical scores of >5 on a cumulative clinical assessment (0-72) of all digits and paws each graded on a scale of 0 to 3 where 0=normal, 1=slight swelling and/or erythema, 2=extensive swelling and/or erythema, and 3=joint distortions and/or rigidity.

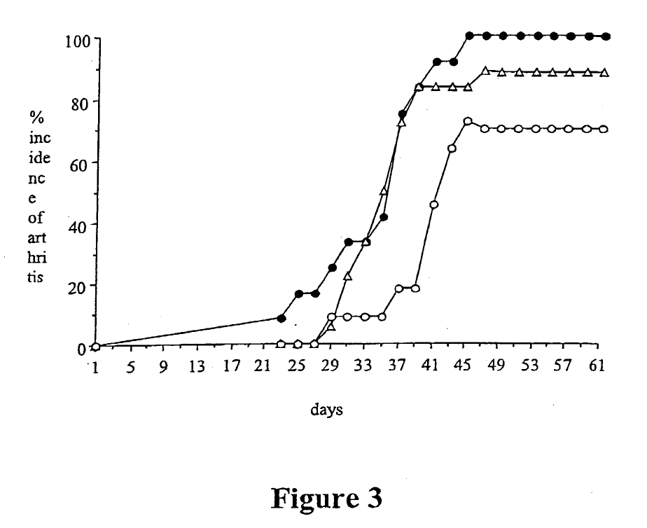

Vegfb knockout mice displayed delayed onset and reduced incidence of disease. The results are shown in FIG. 3.

### EXAMPLE 4

### Development of Rheumatoid Arthritis in Male Vegfb Knockout Mice

Male Vegfb^{+/+} and Vegfb^{-/-} mice (6-8 weeks of age) were immunized with chick CII (100 μg) in CFA containing 2.5 mg/ml heat inactivated M. tuberculosis on day 1 followed by a booster injection of chick CII (100 μg) in IFA on day 8. Disease severity was calculated from day 23 onwards by cumulative clinical assessment (0-72) of all digits and paws, each graded on a scale of 0 to 3 where 0=normal, 1=slight swelling and/or erythena, 2=extensive swelling and/or erythema, and 3=joint distortions and/or rigidity.

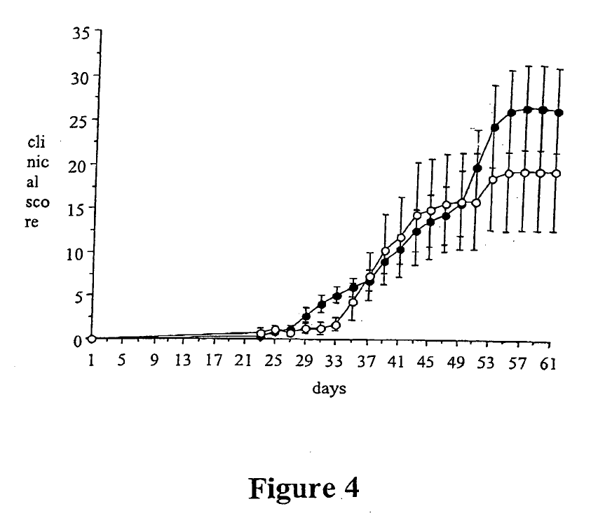

Male Vegfb deficient mice displayed delayed onset and reduced clinical severity at disease onset but not later stages in contrast to female mice. The significance of differences between experimental groups was analyzed using the alternate Welch t-test. Differences in means were considered significant if P<0.05. This is indicated by an “*” in FIG. 4.

### EXAMPLE 5

### Incidence of Rheumatoid Arthritis in Male Vegfb Knockout Mice

Male Vegfb^{+/+} and Vegfb^{-/-} mice (6-8 weeks of age) were immunized to chick CII (100 μg) in CFA containing 2.5 mg/ml heat inactivated M. tuberculosis on day 1 followed by a booster injection of chick CII (100 μg) in IFA on day 8. Disease incidence was determined as the percentage of animals exhibiting clinical scores of >5 on a cumulative clinical assessment (0-72) of all digits and paws each graded on a scale of 0 to 3 where 0=normal, 1=slight swelling and/or erythemna, 2=extensive swelling and/or erythema, and 3=joint distortions and/or rigidity.

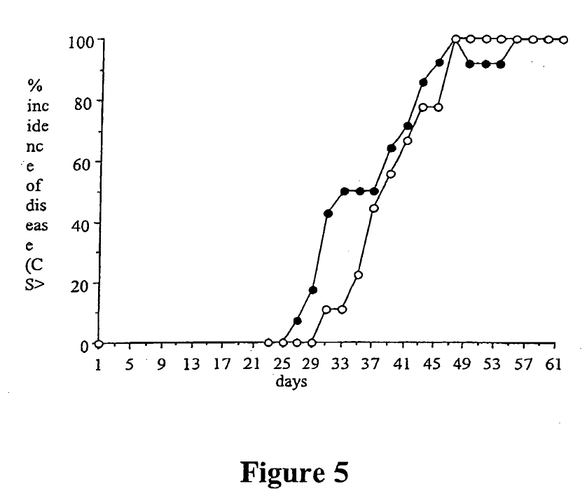

Male Vegfb deficient mice displayed delayed onset and reduced clinical severity at disease onset but not later stages in contrast to female mice. The results are shown in FIG. 5.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

Bibliography

Aase et al. (1999) Dev. Dyn. 215:12-25

Bellomo et al. (2000) Circ. Res. 86:E29-35

Bonner and Laskey (1974) Eur. J. Biochem. 46:83

Grimmond et al. (1996) Genome Res. 6:124-131

Makinen et al. (1999) J. Biol. Chem. 274:21217-21222

Marmur and Doty (1962) J. Mol. Biol. 5:109

Mountford et al. (1994) Proc. Natl. Acad. Sci. U.S.A. 91:4303-4307

Olofsson et al. (1999) Curr. Opin. Biotechnol. 10:528-535

Paavonen et al. (1996) Circulation 93:1079-1082

Sambrook et al. (1990) Molecular Cloning—A Laboratory Manual, Cold Spring Harbor Laboratory, Cold Spring Harbor, N.Y.

Song et al. (1987) Proc. Nat'l. Acad. Sci. USA 84:6820-6824

Southern, P. and Berg, P. (1982) J. Mol. Ap9pl. Genet. 1:327-341

Te Riele et al. (1990) Nature 348:649-651

Thomas et al. (1986) Cell 44:419-428

Thomas, K. and Capechhi, M. (1987) Cell 51:503-512

Townson et al. (1996) Biochem. Biophys. Res. Comm. 220:922-928

1. A method for the treatment and/or prophylaxis of a disease condition in a subject wherein said disease condition is an autoimmune condition exacerbated or otherwise facilitated by the presence of a growth factor or cytokine or expressible genetic material encoding a growth factor or cytokine and/or genetic material which facilitates the expression of said first mentioned genetic material said method comprising reducing or inhibiting the level or activity of the growth factor or cytokine or reducing or inhibiting the expression or function of genetic material encoding said growth factor or cytokine for a time and under conditions sufficient to delay onset of or to otherwise ameliorate the symptoms of said disease condition.

2. A method according to claim 1 wherein the disease condition is rheumatoid arthritis, ankylosing spondylitis, acute anterior uveitis, Goodpastures's syndrome, multiple sclerosis, Graves' disease, myasthenia gravis, systemic lupis erythematosus, insulin-dependent diabetes mellitus, pemphigus vulgaris, Hashimoto's thyroiditis, autoimmune hemolytic anemia, autoimmune thrombocytopenia purpura, acute rheumatic fever, subacute bacterial endocarditis, mixed essential cryoglobulinemia, experimental, autoimmune encephalomyelitis (EAE), hypoglycemia and cold agglutinin disease.

3. A method according to claim 2 wherein the disease condition is rheumatoid arthritis or a related condition.

4. A method according to claim 3, wherein the disease condition is rheumatoid arthritis.

5. A method according to claim 1 or 2 or 3 or 4 wherein the growth factor or cytokine is VEGF-B or a homologue thereof.

6. A method according to claim 5 wherein the VEGF-B homologue is a splice variant.

7. A method for the treatment and/or prophylaxis of rheumatoid arthritis or a related condition in a subject wherein said rheumatoid arthritis or related condition is a condition exacerbated or otherwise facilitated by the presence of a growth factor or cytokine or expressible genetic material encoding a growth factor or cytokine and/or genetic material which facilitates the expression of said first mentioned genetic material said method comprising reducing or inhibiting the level or activity of the growth factor or cytokine or reducing or inhibiting the expression or function of said genetic material encoding said growth factor or cytokine for a time and under conditions sufficient to delay onset of or otherwise ameliorate the symptoms of said rheumatoid arthritis or related condition.

8. A method according to claim 7 wherein the growth factor or cytokine is VEGF-B or a homologue thereof.

9. A method according to claim 8 wherein the VEGF-B homologue is a splice variant.

10. A method according to claim 1 or 7 wherein the subject is a human.

11. A method for the prophylaxis and/or treatment of an autoimmune condition or a related condition, said method comprising reducing the level or activity of VEGF-B or a functional or structural equivalent thereof or reducing or inhibiting the function of genetic material encoding VEGF-B or which facilitates expression of VEGFB or its homologue for at time and under conditions sufficient to reduce onset of or otherwise ameliorate the symptoms of an autoimmune disease or a related condition.

12. A method according to claim 11 wherein the autoimnune condition is rheumatoid arthritis, ankylosing spondylitis, acute anterior uveitis, Goodpastures's syndrome, multiple sclerosis, Graves' disease, myasthenia gravis, systemic lupis erythematosus, insulin-dependent diabetes mellitus, pemphigus vulgaris, Hashimoto's thyroiditis, autoimmune hemolytic anemia, autoimmune thrombocytopenia purpura, acute rheumatic fever, subacute bacterial endocarditis, mixed essential cryoglobulinemia, experimental autoimmune encephalomyelitis (EAE), hypoglycemia and cold agglutinin disease.

13. A method according to claim 12 wherein the autoimmune condition is rheumatoid arthritis or a related condition.

14. A method according to claim 13 wherein the autoimmune condition is rheumatoid arthritis.

15. A composition comprising an antagonist of a VEGF-B or VEGFB or VEGFB homologue and one or more pharmaceutically acceptable carriers and/or diluents for use in the prophylaxis and/or treatment of an autoimmune condition.

16. A composition according to claim 15 wherein the autoimmune condition is rheumatoid arthritis or a related condition.

17. A composition according to claim 16 wherein the autoimmune condition is rheumatoid arthritis.

18. Use of a VEGF-B level- or activity-inhibiting or antagonizing molecule in the manufacture of a medicament for the treatrnent of an autoimmune condition.

19. Use of a VEGFB- or VEGFB homologue- or associated regulatory sequence-expression inhibiting or antagonizing molecule in the manufacture of a medicament for the treatment of an autoimmune condition.

20. Use according to claim 18 or 19 wherein the autoimmune condition is rheumatoid arthritis, ankylosing spondylitis, acute anterior uveitis, Goodpastures's syndrome, multiple sclerosis, Graves' disease, myasthenia gravis, systemic lupis erythematosus, insulin-dependent diabetes mellitus, pemphigus vulgaris, Hashimoto's thyroiditis, autoimmune hemolytic anemia, autoimmune thrombocytopenia purpura, acute rheumatic fever, subacute bacterial endocarditis, mixed essential cryoglobulinemia, experimental autoimmune encephalomyelitis (EAE), hypoglycemia and cold agglutinin disease.

21. Use according to claim 20 wherein the autoimmune condition is rheumatoid arthritis or a related condition.

22. Use according to claim 21 wherein the autoimmune condition is rheumatoid arthritis.

23. A genetically modified animal wherein said animal produces a greater amount of a growth factor or cytokine relative to a non-genetically modified animal of the same species wherein said animal has a predisposition for the development of an autoimmune condition.

24. A genetically modified animal according to claim 23 wherein the animal is a mouse, rat, guinea pig, rabbit, pig, sheep or goat.

25. A genetically modified animal according to claim 24 wherein the animal is a mouse or rat.

26. A genetically modified animal according to claim 25 wherein the animal is a mouse.

27. A genetically modified animal according to any one of claims 23 to 26 wherein the autoimmune condition is rheumatoid arthritis, ankylosing spondylitis, acute anterior uveitis, Goodpastures's syndrome, multiple sclerosis, Graves' disease, myasthenia gravis, systemic lupis erythematosus, insulin-dependent diabetes mellitus, pemphigus vulgaris, Hashimoto's thyroiditis, autoimmune hemolytic anemia, autoimmune thrombocytopenia purpura, acute rheumatic fever, subacute bacterial endocarditis, mixed essential cryoglobulinemia, experimental autoimmune encephalomyelitis (EAE), hypoglycemia and cold agglutinin disease.

28. A genetically modified animal according to claim 27 wherein the autoimmune condition is rheumatoid arthritis.

29. A genetically modified mouse wherein said animal produces a greater amount of a growth factor or cytokine relative to a non-genetically modified mouse of the same species wherein said mouse has a predisposition for the development of an autoimmune condition.

30. A genetically modified mouse according to claims 29 wherein the autoimmune condition is rheumatoid arthritis, ankylosing spondylitis, acute anterior uveitis, Goodpastures's syndrome, multiple sclerosis, Graves' disease, myasthenia gravis, systemic lupis erythematosus, insulin-dependent diabetes mellitus, pemphigus vulgaris, Hashimoto's thyroiditis, autoimmnune hemolytic anemia, autoimmune thrombocytopenia purpura, acute rheumatic fever, subacute bacterial endocarditis, mixed essential cryoglobulinemia, experimental autoimmune encephalomyelitis (EAE), hypoglycemia and cold agglutinin disease.

31. A genetically modified animal according to claim 30 wherein the autoimmune condition is rheumatoid arthritis or related condition.

32. A genetically modified animal according to claim 31 wherein the autoimmune condition is rheumatoid arthritis..

33. A genetically modified animal wherein said animal is substantially incapable of producing a growth factor or cytokine relative to a non-genetically modified animal of the same species wherein said animal has a reduced onset or reduced clinical severity of an autoimmune condition.

34. A genetically modified animal according to claim 33 wherein the animal is a mouse, rat, guinea pig, rabbit, pig, sheep or goat.

35. A genetically modified animal according to claim 34 wherein the animal is a mouse or rat.

36. A genetically modified animal according to claim 35 wherein the animal is a mouse.

37. A genetically modified animal according to any one of claims 33 to 36 wherein the autoimmune condition is rheumatoid arthritis, ankylosing spondylitis, acute anterior uveitis, Goodpastures's syndrome, multiple sclerosis, Graves' disease, myasthenia gravis, systemic lupis erythematosus, insulin-dependent diabetes mellitus, pemphigus vulgaris, Hashimoto's thyroiditis, autoimmune hemolytic anemia, autoimmune thrombocytopenia purpura, acute rheumatic fever, subacute bacterial endocarditis, mixed essential cryoglobulinemia, experimental autoimmune encephalomyelitis (EAE), hypoglycemia and cold agglutinin disease.

38. A genetically modified animal according to claim 37 wherein the autoimmune condition is rheumatoid arthritis or a related condition.

39. A genetically modified animal according to claim 38 wherein the autoimmune condition is rheumatoid arthritis.

40. A genetically modified mouse wherein said mouse is substantially incapable of producing a growth factor or cytokine relative to a non-genetically modified mouse of the same species wherein said mouse has a reduced onset or reduced clinical severity of an autoimmune condition.

41. A genetically modified animal according to claims 40 wherein the autoimmune condition is rheumatoid arthritis, ankylosing spondylitis, acute anterior uveitis, Goodpastures's syndrome, multiple sclerosis, Graves' disease, myasthenia gravis, systemic lupis erythematosus, insulin-dependent diabetes mellitus, pemphigus vulgaris, Hashimoto's thyroiditis, autoimmune hemolytic anemia, autoimmune thrombocytopenia purpura, acute rheumatic fever, subacute bacterial endocarditis, mixed essential cryoglobulinemia, experimental autoimmune encephalomyelitis (EAE), hypoglycemia and cold agglutinin disease.

42. A genetically modified animal according to claim 40 wherein the autoimmune condition is rheumatoid artritis.

43. A targeting vector useful for inactivating a gene encoding a growth factor or cytokine, said targeting vector comprising two segments of genetic material encoding said growth factor or cyktokine flanking a positive selectable marker wherein when said targeting vector is transfected into embryonic stem (ES) cells and the marker selected, an ES cell is generated in which the Vegfb gene encoding said growth factor or cytokine is inactivated by homologous recombination.

44. A targeting vector according to claim 43 wherein the ES cells are from mice, rats, guinea pigs, pigs, sheep or goats.

45. A targeting vector according to claim 44 wherein the ES cells are from mice.

46. A targeting vector useful for inactivating a gene encoding VEGF-B, said targeting vector comprising two segments of genetic material encoding VEGF-B flanking a positive selectable marker wherein when said targeting vector is transfected into embryonic stem (ES) cells and the marker selected, an ES cell is generated in which the Vegfb gene is inactivated by homologous recombination.

47. A targeting vector according to claim 46 wherein the ES cells are from mice, rats, guinea pigs, pigs, sheep or goats.

48. A targeting vector according to claim 47 wherein the ES cells are from mice.

49. Use of a targeting vector according to claim 43 or 46 in the manufacture of a genetically modified animal substantially incapable of producing VEGF-B.
